(19) United States
(12) Plant Patent Application Publication
(10) Pub. No.: US 2003/0009809 P1
(43) Pub. Date: Jan. 9, 2003

(54) Chrysanthemum plant named 'Vulcan Time'

(76) Inventor: Mark Roland Boeder, Den Haag (NL)

Correspondence Address: Mark K. Dickson, HOWREY SIMON ARNOLD & WHITE, LLP, 750 Bering Drive, Houston, TX, 77057-2198, US

(21) Appl. No.: 09/900,234

(22) Filed: Jul. 6, 2001

(51) Int. Cl. A01H 5/00

(52) U.S. Cl. PLT/298

(57) ABSTRACT

A new variety of Chrysanthemum plant named ‘Vulcan Time,’ having red single type flowers, a slight domed capitulum form and an upright growth habit.

### BACKGROUND OF THE INVENTION

The present invention comprises a new and distinct cultivar of Chrysanthemum botanically known as Dendranthema grandiflora, and referred to by the cultivar name ‘Vulcan Time.’

‘Vulcan Time’, identified as 20247-0, originated from a cross made by Mark R. Boeder in a controlled breeding program at Chichester, West Sussex, United Kingdom. The female parent of the new variety is an undistributed, unpatented seedling identified as P551B2 and the male parent is the undistributed, unpatented seedling identified as P341B7. The new variety ‘Vulcan Time’ has been asexually reproduced by vegetative cuttings at Chichester, West Sussex, United Kingdom and the distinguishing characteristics are retained through successive generations of asexual reproduction.

### BRIEF SUMMARY OF THE INVENTION

‘Vulcan Time’ is a pot type of Chrysanthemum plant, having a flat capitulum form and red single type flowers. The age of ‘Vulcan Time’ in the photo is 60 days from the onset of short days.

### Comparison with Parent

Plants of the new Chrysanthemum variety ‘Vulcan Time’ are similar to the parent seedling P341B7 in plant habitat and growth rate. In side-by-side comparisons at Chichester, West Sussex, United Kingdom, under commercial practice, plants of the new Chrysanthemum variety ‘Vulcan Time’ differed from plants of the parent seedling P341B7 in the following characteristics.

1. The new variety ‘Vulcan Time’ produces paler red flowers whereas the parent seedling P341B7 produces dark red flowers.

2. Plants of the new variety ‘Vulcan Time’ have a faster response time than plants of the parent seedling P341B7.

3. Plants of the new variety ‘Vulcan Time’ have paler green foliage than plants of the parent seedling P341B7.

### Comparison with Other Varieties

Plants of the new Chrysanthemum variety ‘Vulcan Time’ are similar to the cultivar ‘Rage’ U.S. Plant Pat. No. 8,770 in plant habitat and growth rate. However, in side-by-side comparisons at Chichester, West Sussex, United Kingdom, under commercial practice, plants of the new Chrysanthemum variety ‘Vulcan Time’ differed from plants of the cultivar ‘Rage’ in the following characteristics.

1 Plants of the new variety ‘Vulcan Time’ have a faster response time than plants of the variety ‘Rage.’

2 Plants of the new variety ‘Vulcan Time’ have larger flowers than plants of the cultivar ‘Rage.’

### BRIEF DESCRIPTION OF ILLUSTRATIONS

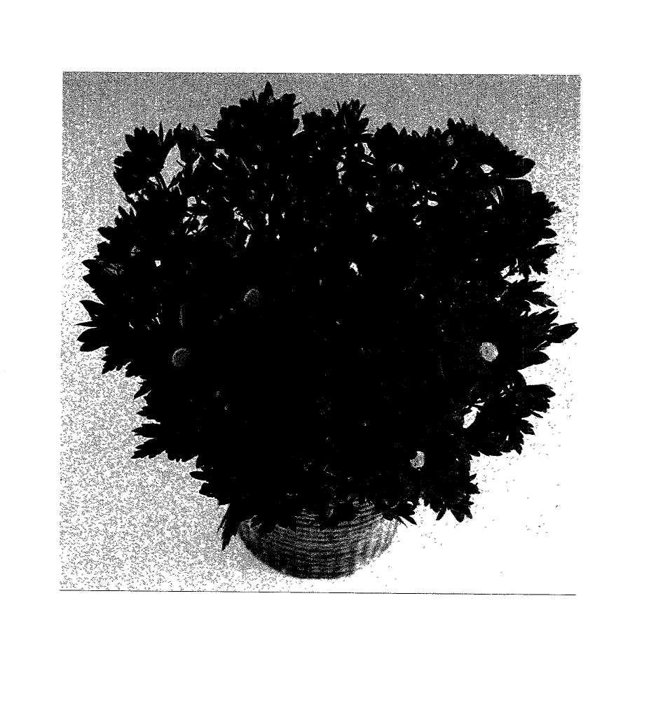

FIGURE 1 is a whole plant view of the new variety ‘Vulcan Time’ grown in a pot. The colors shown are as true as possible within the usual limits of this kind of illustration.

### DETAILED BOTANICAL DESCRIPTION

The following description of the new Chrysanthemum variety ‘Vulcan Time’ is of plants grown a greenhouse at Chichester, West Sussex, United Kingdom in the month of June. The cultivar has not been observed under all possible environmental conditions. The phenotype may vary significantly with variations in the environment such as temperature, length of day and light intensity, without any variance in genotype. The commercial classification of the new variety is a pot Chrysanthemum.

Plants of the new variety have been grown successfully under temperature conditions averaging about 18° C. at night and about 24° C. to 25° C. during the day under light conditions of about 5,000 to 6,000 foot candles. The plants respond well to the use of growth retardant, such as three B9 treatments at about 2,500 ppm. To produce a commercial product the plants may be pinched once. The typical container size for commercial growth is 14 cm. It has been observed that the shelf life of the new variety is about 21 days with a response time of about 8 weeks.

The following description is with respect to a plant produced as a pot Chrysanthemum. In the description of this new Chrysanthemum variety, color values have been taken from The Royal Horticultural Society Colour Chart (R.H.S.C.C.).

### Plant

Plant type: pot

Habit: upright and branching

Height: medium

Width: 32 cm

Branching characteristics (describe): free and spreading

Length of Lateral Branches: 13-17 cm

Number of Breaks from Pinch: 4-6

Stem Color: near 137B

Response Time: near 56 days

Vigor: medium

Shelf Life: near 21 days

Disease (susceptibility or resistance observed): no observations

Growth retardant type and treatment: 3 applications of 2.5 gram/litre at 2 days, 21 days and 28 days after sticking of unrooted cuttings p0 Propagation:

Type.—vegetative propagation via stem cuttings

Time to rooting.—12-14 days with soil temperatures of 18° C.

Rooting habit.—fine and fibrous

### Foliage

Number of Leaves Per Lateral Branch: 9-12

Compound or single: single

Arrangement of Leaves: alternate Shape of Leaf: typically 5 lobed, ovate Size of Leaf: Width (cm): near 4-6 Length (cm): near 6-10 Leaf Apex: acute Base: obtuse Attachment: petioled Aspect: slight undulating Margin: palmately lobed Surface characteristics: Top: slightly pubescent Bottom: pubescent

Petiole: Color: near 137B Length: near 2.5 cm

Venation (describe): net, prominent midvein at under side Color: upper side: near 144B under side: near 144B

Color: Mature Leaf, upper side: near 137B; underside: near 138B Young Leaf, upper side: near 137A; under side: near 137C

### Flower

Flower type: single

Number of blossoms per branch: 3-5

Inflorescence form: pyramidal

Depth of fully expanded blossoms.—near 2.0 cm

Diameter of fully expanded blossoms.—6-8 cm

Sepals:

Number.—20-25

Color.—near 137B

Length.—0.7-1.2 cm

Width.—0.2-0.3 cm

Texture/Appearance.—pubescent

Pedicel length is more or less equal over all flowers and between 4.5 and 5.5 cm

Length.—4.5-5.5 cm

Color.—near 137B

Surface.—pubescent

Habit.—slightly undulating

Strength.—medium

Ray Florets:

Form/shape.—straight

Texture/appearance.—matte

Number per flower.—35-45

Length.—3.5-4.5 cm; Width: 0.6-1.0 cm

Apex.—rounded or dentate

Base.—tapered

Margin.—entire

Disc florets:

Form/shape.—cylindrical

Texture/appearance.—shiny

Number per flower.—170-250

Length.—near 0.4-0.6 cm; Width: <0.1 cm

Diameter of disc.—1.0-1.4 cm

Fragrance: faint

Flower Bud (at onset of color):

Length.—near 0.8 cm

Diameter.—near 1.1 cm

Form/shape.—globular

General flower color:

1. Ray florets, upper side:

Immature.—near 46A

Mature.—near 45B

Older/Fading.—near 44B

2. Ray florets, under side:

Immature.—near 33C

Mature.—near 33C

Older/fading.—near 33D

3. Disc florets:

Immature.—near 151B

Mature.—near 6A

Older/fading.—near 6A

4. Bud: near 138B

Flower progression with age: Flowers fading from red to bronze/orange with age.

### Reproductive Organs

Ray Florets: Pistillate

Stigma color.—near 6C

Stigma shape.—forked

Style color.—near 6C

Style length.—near 0.4 cm

Disc Florets: Pistillate

Stigma color.—near 6C

Stigma shape.—forked

Style color.—near 6C

Style length.—near 0.1 cm

Staminate.—

Stamen Number.—5

What is claimed is:

1. A new and distinct variety of Chrysanthemum plant, substantially as described and illustrated herein.
